(19) United States
(12) Patent Application Publication
(10) Pub. No.: US 2013/0177951 A1
(43) Pub. Date: Jul. 11, 2013

(54) CHEMO-ENZYMATIC PROCESS FOR PREPARING QUATERNARY AMMONIUM ESTERS

(71) Applicants: Christopher Harlan Burk, Gray, TN (US); Neil Warren Boaz, Kingsport, TN (US); Stephanie Kay Clendennen, Kingsport, TN (US)

(72) Inventors: Christopher Harlan Burk, Gray, TN (US); Neil Warren Boaz, Kingsport, TN (US); Stephanie Kay Clendennen, Kingsport, TN (US)

(73) Assignee: EASTMAN CHEMICAL COMPANY, Kingsport, TN (US)

(21) Appl. No.: 13/345,028

(22) Filed: Jan. 6, 2012

(51) Int. Cl. C12P 13/00

(52) U.S. Cl. 435/128

(57) ABSTRACT

A process for producing surface-active quaternary ammonium esters is provided. These esters are advantageously prepared in high yield and purity by a chemo-enzymatic process. These compounds have excellent surfactant properties.

### FIELD OF THE INVENTION

The invention generally relates to a process for preparing quaternary ammonium ester compounds, and in particular, to a chemo-enzymatic process for preparing such compounds.

### BACKGROUND OF THE INVENTION

There is an increasing industrial and societal need for chemical processes that reduce or eliminate use of organic solvents and irritants, that employ reagents derived from a natural source, and that reduce energy consumption and waste. This is of urgent interest in consumer-facing industries such as personal, household, and laundry care. One class of materials that might be approached in a “greener” manner are surfactants. In particular, there is a need for a more environmentally-friendly process to produce quaternary ammonium esters (“ester quats”). Ester quats are cationic surfactants primarily used in the fabric care industry. Additionally, these surfactants are used in personal care applications such as hair care and skin care.

Ester quats generally refer to quaternary ammonium compounds in which one or more fatty acid chains (C₆-C₁₈) are connected to the ammonium backbone by ester linkages. Birkhan et al. (U.S. Pat. No. 5,180,508) describe the advantages that these compounds have over dialkyldimethyl ammonium salts. The ester linkages in these compounds improve their biodegradability as well as their cold-water dispersibility. Two commonly used ester quats are diethyl ester dimethyl ammonium chloride (DEEDMAC) and diethyl ester hydroxyethyl methyl ammonium methyl sulfate (DEEHAMS).

Ester quats are commonly produced by a two-step chemical process based on fatty acids derived from coconut oil, palm kernel oil, tallow (hydrogenated or nonhydrogenated), etc. Vegetable-derived oils are preferred for personal care applications. The two-step process involves esterifying the fatty acid with an alkanolamine, followed by quaternization with a quaternizing agent of choice.

For example, Toney et al. (U.S. Pat. No. 5,523,433) describe a process for the production of DEEDMAC. In this process, the fatty acid is esterified with diethanolamine in the presence of a homogeneous acid catalyst at temperatures of about 180-200° C., followed by quaternization with methyl chloride.

Contet et al. (U.S. Pat. No. 5,750,492) describe the preparation of DEEHAMS. This process involves esterifying fatty acids with triethanolamine in the presence of a homogeneous catalyst at or above temperatures of 140° C., followed by quaternization with dimethyl sulfate.

Chang (EP 0 309 052 B1) describes another process for producing ester quats. This process involves esterifying fatty acid chlorides with alkanolamines in chlorinated solvents using triethylamine as an acid acceptor followed by quaternization.

Hoffman et al. (U.S. Pat. No. 4,339,391) describe yet another process for producing various ester quats. This process involves the direct esterification of fatty acids with an alkyl diethanolamine in the presence of a homogeneous catalyst at temperatures greater than 150° C. Subsequently, the diesteramine is quaternized with an alkyl halide.

The aforementioned processes use fatty acids and fatty acid chlorides as starting materials. Producing fatty acids and fatty acid chlorides from their respective triglycerides involves a fat-splitting process that requires temperatures in excess of 250° C., pressures from 700-900 psig (4.8-6.2 MPa), and toxic reagents. Typically, the esterification processes mentioned above are operated at temperatures up to 200° C. (with or without vacuum) to drive off water and achieve high conversion to the ester-amine intermediate. Processes using a homogeneous catalyst must include a wash step to remove the catalyst. Thus, ester quats prepared under mild conditions with less energy consumption and less production of waste and/or by-products, would be highly desirable.

While not directed to ester quats, Clendennen and Boaz (U.S. Pat. No. 7,667,067) describe a biocatalytic process for preparing dimethylaminoethanol fatty acid esters.

Accordingly, there is a need in the art for a process to produce ester quats under mild conditions and in high yield. Ideally, such a process would take place at lower temperatures, with fewer processing steps and by-products, and with less environmental impact.

The present invention addresses this need as well as others that will become apparent from the following description and the appended claims.

### SUMMARY OF THE INVENTION

The invention is as set forth in the appended claims.

Briefly, the invention provides a process for preparing quaternary ammonium ester compounds. The process comprises (a) contacting a fatty acid ester with an amino-alcohol in the presence of an enzyme under conditions effective to form an amino-ester compound, and (b) contacting the amino-ester compound with a quaternizing agent selected from alkyl halides, alkyl sulfates, alkyl phosphates, and alkyl carbonates under conditions effective to produce a quaternary ammonium ester compound.

### DETAILED DESCRIPTION OF THE INVENTION

The invention provides a chemo-enzymatic process for preparing quaternary ammonium ester compounds. Preferably, the ester quats have the general formula 1:

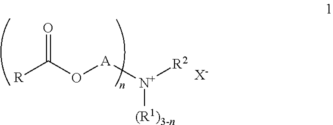

wherein

R is selected from substituted and unsubstituted, branched- and straight-chain, saturated, unsaturated, and polyunsaturated C₁-C₂₂ hydrocarbyl, substituted and unsubstituted C₃-C₈ cycloalkyl, substituted and unsubstituted C₆-C₂₀ carbocyclic aryl, and substituted and unsubstituted C₄-C₂₀ heterocyclic wherein the heteroatoms are selected from sulfur, nitrogen, and oxygen, or mixtures thereof;

R¹ and R², which may be the same or different, are independently selected from substituted or unsubstituted straight- or branched-chain C₁-C₂₂ alkyl, alkenyl, dienyl, trienyl, and C₃-C₈ cycloalkyl groups wherein the branching and/or substitution of R¹ and R² may connect to form a ring;

A is selected from substituted and unsubstituted, branched- and straight-chain, saturated, unsaturated, and polyunsaturated C₁-C₁₀ divalent hydrocarbyl, substituted and unsubstituted C₃-C₈ cycloalkylene, substituted and unsubstituted C₆-C₁₀ carbocyclic arylene, and substituted and unsubstituted C₄-C₁₀ divalent heterocyclic wherein the heteroatoms are selected from sulfur, nitrogen, and oxygen;

X⁻ is an anion of a quaternizing agent;

n is an integer from 1 to 3.

Preferred species are compounds denoted by the general formula 1 wherein

R is selected from substituted and unsubstituted, branched- and straight-chain saturated C₁-C₂₂ alkyl, substituted and unsubstituted, branched- and straight-chain C₂-C₂₂ alkenyl, substituted and unsubstituted, branched- and straight-chain C₄-C₂₂ dienyl, substituted and unsubstituted C₃-C₈ cycloalkyl, substituted and unsubstituted C₆-C₂₀ carbocyclic aryl, and substituted and unsubstituted C₄-C₂₀ heteroaryl;

R¹ and R² are selected from straight or branched chain C₁-C₂₂ alkyl or alkenyl;

A is selected from substituted and unsubstituted, branched and straight chain C₁-C₈ alkylene, branched- and straight-chain saturated C₂-C₈ alkenylene, substituted and unsubstituted C₃-C₈ cycloalkylene, substituted and unsubstituted C₈-C₁₀ carbocyclic arylene, substituted and unsubstituted C₄-C₁₂ divalent heterocyclic, and mixtures thereof;

X⁻ is a halide, methosulfate, ethosulfate, carbonate, methyl carbonate, or phosphate; and

n is an integer from 1 to 3.

The saturated, unsaturated, and polyunsaturated alkyl, and cycloalkyl groups, which may be represented by R, may be straight- or branched-chain hydrocarbon radicals containing up to about 22 carbon atoms and may be substituted, for example, with one to five groups selected from C₁-C₆-alkoxy, carboxyl, amino, C₁-C₁₅ aminocarbonyl, C₁-C₁₅ amido, cyano, C₂-C₆-alkoxycarbonyl, C₂-C₆-alkanoyloxy, hydroxy, aryl, heteroaryl, thiol, thioether, C₂-C₁₀ dialkylamino, C₃-C₁₅ trialkylammonium, and halogen.

The terms “C₁-C₆-alkoxy,” “C₂-C₆-alkoxycarbonyl,” and “C₂-C₆-alkanoyloxy” are used to denote radicals corresponding to the structures —OR³, —CO₂R³, and —OCOR³, respectively, wherein R³ is C₁-C₆-alkyl or substituted C₁-C₆-alkyl.

The terms “C₁-C₁₅ aminocarbonyl” and “C₁-C₁₅ amido” are used to denote radicals corresponding to the structures —NHCOR⁴ and —CONHR⁴, respectively, wherein R⁴ is C₁-C₁₅-alkyl or substituted C₁-C₁₅-alkyl.

The term “C₃-C₈-cycloalkyl” is used to denote a saturated, carbocyclic hydrocarbon radical having three to eight carbon atoms.

The alkyl, alkenyl, dienyl, and trienyl groups, which may be represented by R¹ and R², may be straight- or branched-chain hydrocarbon radicals containing up to about 22 carbon atoms and may be substituted, for example, with one to three groups selected from C₁-C₂₀-hydrocarbyloxy, carboxyl, amino, C₁-C₁₅ aminocarbonyl, C₁-C₁₅ amido, cyano, C₂-C₂₀-hydrocarbyloxycarbonyl, C₂-C₂₀-hydrocarbanoyloxy, hydroxy, aryl, heteroaryl, thiol, thioether, C₂-C₁₀ dialkylamino, C₃-C₁₅ trialkylammonium, and halogen.

The terms “C₁-C₂₀-hydrocarbyloxy,” “C₂-C₂₀-hydrocarbyloxycarbonyl,” and “C₂-C₂₀-hydrocarbanoyloxy” are used to denote radicals corresponding to the structures —OR⁵, —CO₂R⁵, and —OCOR⁵, respectively, wherein R⁵ is an alkyl or alkenyl or substituted alkyl or alkenyl group containing up to 20 carbon atoms.

The terms “C₁-C₁₅ aminocarbonyl” and “C₁-C₁₅ amido” are as described above.

The term “C₃-C₈-cycloalkyl” is as described above.

In the case where there is more than one R¹ group, they may be the same or different.

The divalent hydrocarbyl radicals, which may be represented by A, may be straight- or branched-chain saturated, unsaturated, and polyunsaturated alkylene and cycloalkylene groups containing up to about 6 carbon atoms and may be substituted, for example, with one to five groups selected from C₁-C₂₀-hydrocarbyloxy, carboxyl, amino, C₁-C₁₅ aminocarbonyl, C₁-C₁₅ amido, cyano, C₂-C₂₀-hydrocarbyloxycarbonyl, C₂-C₂₀-hydrocarbanoyloxy, hydroxy, aryl, heteroaryl, thiol, thioether, C₂-C₁₀ dialkylamino, C₃-C₁₅ trialkylammonium, and halogen.

The terms “C₁-C₂₀-hydrocarbyloxy,” “C₂-C₂₀-hydrocarbyloxycarbonyl,” and “C₂-C₂₀-hydrocarbanoyloxy” are as described above.

The terms “C₁-C₁₅ aminocarbonyl” and “C₁-C₁₅ amido” are as described above.

The aryl groups, which R may represent (or any aryl substituents), may include phenyl, naphthyl, or anthracenyl and phenyl, naphthyl, or anthracenyl substituted with one to five substituents selected from C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, C₂-C₆-alkoxycarbonyl, C₂-C₆-alkanoylamino and —OR⁶, —S—R⁶, —SO₂—R⁶, —NHSO₂R⁶, and —NHCO₂R⁶, wherein R⁶ is phenyl, naphthyl, or phenyl or naphthyl substituted with one to three groups selected from C₁-C₆-alkyl, C₆-C₁₀ aryl, C₁-C₆-alkoxy, and halogen.

The arylene groups, which A may represent, may include phenylene, naphthylene, or anthracenylene and phenylene, naphthylene, or anthracenylene substituted with one to four substituents selected from C₁-C₆-alkyl, substituted C₁-C₆-alkyl, C₆-C₁₀ aryl, substituted C₆-C₁₀ aryl, C₁-C₆-alkoxy, halogen, carboxy, cyano, C₁-C₆-alkanoyloxy, C₁-C₆-alkylthio, C₁-C₆-alkylsulfonyl, trifluoromethyl, hydroxy, —_{OR}⁶, —S—R⁶, —SO₂—R⁶, C₂-C₆-alkoxycarbonyl, C₂-C₆-alkanoylamino and —NHSO₂R⁶ and —NHCO₂R⁶, wherein R⁶ is phenyl, naphthyl, or phenyl or naphthyl substituted with one to three groups selected from C₁-C₆-alkyl, C₆-C₁₀ aryl, C₁-C₆-alkoxy, and halogen.

The heterocyclic groups, which R may represent (or any heteroaryl substituents), include 5- or 6-membered rings containing one to three heteroatoms selected from oxygen, sulfur, and nitrogen. Examples of such heterocyclic groups are pyranyl, oxopyranyl, dihydropyranyl, oxodihydropyranyl, tetrahydropyranyl, thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, pyridyl, pyrimidyl, benzoxazolyl, benzothiazolyl, benzimidazolyl, indolyl, and the like. The heterocyclic radicals may be substituted, for example, with up to three groups such as C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino. The heterocyclic radicals also may be substituted with a fused ring system, e.g., a benzo or naphtho residue, which may be unsubstituted or substituted, for example, with up to three of the groups set forth in the preceding sentence.

The divalent heterocyclic groups, which A may represent, include 5- or 6-membered rings containing one to three heteroatoms selected from oxygen, sulfur, and nitrogen. Examples of such heterocyclic groups are pyranyl, oxopyranyl, dihydropyranyl, oxodihydropyranyl, tetrahydropyranyl, thienyl, furyl, pyrrolyl, imidazolyl, pyrazolyl, thiazolyl, isothiazolyl, oxazolyl, isoxazolyl, triazolyl, thiadiazolyl, oxadiazolyl, tetrazolyl, pyridyl, pyrimidyl, benzoxazolyl, benzothiazolyl, benzimidazolyl, indolyl, and the like. The heterocyclic radicals may be substituted, for example, with up to three groups such as C₁-C₆-alkyl, C₁-C₆-alkoxy, substituted C₁-C₆-alkyl, halogen, C₁-C₆-alkylthio, aryl, arylthio, aryloxy, C₂-C₆-alkoxycarbonyl, and C₂-C₆-alkanoylamino. The heterocyclic radicals also may be substituted with a fused ring system, e.g., a benzo or naphtho residue, which may be unsubstituted or substituted, for example, with up to three of the groups set forth in the preceding sentence.

The term “halogen” is used to include fluorine, chlorine, bromine, and iodine, and the term “halide” is used to include fluoride, chloride, bromide, and iodide.

Preferred examples of the compounds prepared by the process of the invention include those represented by the general formula 1 wherein R is a C₅ to C₁₇ hydrocarbyl (derived from coconut oil or palm kernel oil), R¹ is methyl or hydroxyethyl, R² is methyl, ethyl, or benzyl, n is 1 to 3, and A is 1,2-ethylene or 1,3-propylene.

The process according to the invention comprises the steps of (a) contacting a fatty acid ester with an amino-alcohol in the presence of an enzyme under conditions effective to form an amino-ester compound, and (b) contacting the amino-ester compound with a quaternizing agent selected from alkyl halides, alkyl sulfates, alkyl phosphates, and alkyl carbonates under conditions effective to produce a quaternary ammonium ester compound.

The fatty acid ester preferably has the general formula 2:

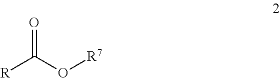

wherein R is as defined above in formula 1, and R⁷ is a C₁-C₆ straight or branched chain alkyl.

The fatty acid esters can be produced by any practical method, including the solvolysis of triglycerides in the presence of a lower alcohol and a base, acid, or enzyme catalyst as is known in the art. The preferred lower alcohols are C₁-C₄ alcohols such as methanol, ethanol, 1-propanol, 2-propanol, 1-butanol, 2-butanol, and isobutanol. A preferred method includes reacting triglycerides (e.g., from coconut or palm kernel oil) with a C₁-C₄ alcohol in the presence of a base such as sodium or potassium hydroxide.

The amino-alcohol reacted with the fatty acid ester preferably has the general formula 3:

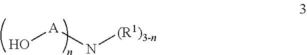

wherein R¹, A, and n are as defined in formula 1. Examples of compounds represented by formula 3 include, but are not limited to, triethanolamine (TEA), methyldiethanolamine (MDEA), N,N-dimethylethanolamine (DMEA), 3-(dimethylamino)-1,2-propanediol (DMAPD), and aminoethylethanolamine (AEEA).

The amino-ester intermediate compound produced in step (a) preferably has the general formula 4:

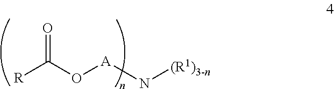

wherein R, R¹, A, and n are as defined in formula 1.

The enzymatic reaction of step (a) may be carried out without added solvent or in the presence of an inert solvent selected from cyclic or acyclic ether solvents, such as diethyl ether, diisopropyl ether, tert-butyl methyl ether, or tetrahydrofuran; aromatic hydrocarbons such as benzene, toluene, or xylene; aliphatic or alicyclic saturated or unsaturated hydrocarbons such as hexane, heptane, cyclohexane, or limonene; halogenated hydrocarbons such as dichloromethane, dichloroethane, dibromoethane, tetrachloroethylene, or chlorobenzene; polar aprotic solvents such as acetonitrile, dimethyl formamide, or dimethyl sulfoxide; or mixtures thereof. Preferably, step (a) is carried out in the absence of added solvent.

Step (a) may be carried out at a temperature ranging from about −100° C. to the boiling point of the solvent, if used. Preferably, step (a) is carried out at a temperature ranging from about 20-80° C., and more preferably from 50-70° C.

The amount of fatty acid ester introduced into the reaction may range from 0.5 to 20 equivalents based on the number of hydroxyl groups on the amino-alcohol. Preferably, the amount of fatty acid ester ranges from 0.5 to 10 equivalents and more preferably from 0.5 to 1.5 equivalents. Using short chain alcohol esters of the fatty acids is beneficial to the success of the enzymatic esterification of the amino-alcohol. Unmodified fatty acids may be used in the enzymatic esterification; however, the acid forms a salt with the amino-alcohol, which tends to limit the efficiency of the reaction.

The enzyme used in step (a) may be a protease, a lipase, or an esterase. Preferred enzymes are lipases. These lipases may be in the form of whole cells, isolated native enzymes, or immobilized on supports. Examples of these lipases include, but are not limited, to Lipase PS (from Pseudomonas sp), Lipase PS-C (from Psuedomonas sp immobilized on ceramic), Lipase PS-D (from Pseudomonas sp immobilized on diatomaceous earth), Lipoprime 50T, Lipozyme TL IM, and Novozym 435 (Candida antarctica lipase B immobilized on acrylic resin).

In addition to the desired amino-ester intermediate, step (a) can produce water or alcohol as by-products. The water and/or alcohol by-products may be removed from the amino-ester intermediate before its introduction into step (b).

The alcohol and/or water by-products can be removed chemically via an alcohol or a water adsorbent (e.g., molecular sieves), or by physical separation of the alcohol or water. Preferably, the by-products are removed by evaporation, e.g., by purging the reaction mixture with an inert gas such as nitrogen, argon, or helium; or by performing the reaction at a reduced pressure, or both, as these conditions can afford >98% conversion of the fatty acid ester to the amino-ester intermediate. The preferred pressure for the enzymatic reaction is from 1 torr (133 Pa) to ambient pressure (101 kPa), more preferably from 10 torr (approx. 1 kPa) to ambient pressure (101 kPa). Any organic solvent that is included in this process may or may not be removed along with the alcohol or water.

Step (b) of the process of the invention comprises reacting the amino-ester intermediate with a quaternizing agent (sometimes called an alkylating agent) to generate the final ester quat product. Suitable quaternizing agents include, but are not limited to, alkyl halides, alkyl sulfates, alkyl phosphates, and alkyl carbonates. The preferred alkyls include those containing 1 to 7 carbon atoms, such as methyl, ethyl, and benzyl. Preferred quaternizing agents include alkyl halides, dimethyl sulfate, or dimethyl carbonate.

Step (b) can be carried out without an added solvent or in an inert solvent selected from water, cyclic or acyclic alcohol solvents such as methanol, ethanol, 1-propanol, 2-propanol, 1-butanol, 2-butanol, isobutanol, ethylene glycol, 1,2-propanediol, or 1,3-propanediol; ketones such as acetone, methyl ethyl ketone, diethyl ketone, methyl propyl ketone, methyl isopropyl ketone, methyl butyl ketone, methyl isobutyl ketone, or methyl amyl ketone; cyclic or acyclic ether solvents such as diethyl ether, diisopropyl ether, tert-butyl methyl ether, or tetrahydrofuran; aromatic hydrocarbons such as benzene, toluene, or xylene, aliphatic or alicyclic saturated or unsaturated hydrocarbons such as hexane, heptane, cyclohexane, or limonene; halogenated hydrocarbons such as dichloromethane, dichloroethane, dibromoethane, tetrachloroethylene, or chlorobenzene; polar aprotic solvents such as acetonitrile, dimethyl formamide, or dimethyl sulfoxide; or mixtures thereof. It is preferred to carry out step (b) in the absence of added solvent. If a solvent is to be used, the preferred solvents are water, alcohols such as isopropanol, ketones such as acetone, or mixtures thereof.

Step (b) may be carried out at a temperature from about −100° C. to the boiling point of the solvent, if present. Preferably, the reaction is carried out at a temperature from about −10 to 150° C., more preferably from 0 to 100° C. The process may be carried out at a pressure from about 1 atmosphere (101 kPa) to 10 atmospheres (1013 kPa).

The amount of quaternizing agent introduced into the reaction may range from 0.5 to 20 equivalents based on the amino-ester. Preferably, the quaternizing agent amount ranges from 0.75 to 10 equivalents, and more preferably from 0.9 to 1.5 equivalents.

The amino-ester intermediate and the ester quat product may be isolated using methods known to those of skill in the art, e.g., extraction, filtration, or crystallization.

Another aspect of the invention is the use of the ester quats of formula 1 as surfactants. The ester quats are useful as surfactants in a number of applications, including fabric care, cosmetic and personal care products, hair care and skin care. Although the primary use is in fabric care, ester quats have also found utility in industrial applications such as paper softening, fertilizer production, ore floatation, and dye dispersion. Ester quats can also be formulated into germicides.

Such product formulations can contain from about 0.001 weight % to about 20 weight %, from about 0.01 weight % to about 15 weight %, or even from about 0.1 weight % to about 10 weight % of the ester quats.

Product formulations of the invention may include other surfactants in addition to the ester quats. These surfactants can include anionic surfactants (such as alcohol ether sulfates, linear alkylbenzene sulfonates, acyl isethionates), cationic surfactants (such as quaternary ammonium salts and fatty amine oxides), and non-ionic surfactants (such as alky polyglycosides, alcohol ethoxylates, and fatty alcanol amides). Such ingredients are known to those of skill in the art.

The compositions of the invention may also contain other ingredients in addition to the ester quats. Such other ingredients are known to those of skill in the art. Many preparations are known in the art, and include formulations containing water, oils and/or alcohols and emollients such as olive oil, hydrocarbon oils and waxes, silicone oils, other vegetable, animal or marine fats or oils, glyceride derivatives, fatty acids or fatty acid esters or alcohols or alcohol ethers, lecithin, lanolin and derivatives, polyhydric alcohols or esters, linear alkoxylated alcohols, soil release agents, wax esters, sterols, phospholipids and the like. These same general ingredients can be formulated into liquids (such as liquid fabric softeners, liquid soaps, shampoos, or body washes), dryer sheets, creams, lotions, gels, or into solid sticks by utilization of different proportions of the ingredients and/or by inclusion of thickening agents such as gums or other forms of hydrophilic colloids.

This invention can be further illustrated by the following examples of preferred embodiments thereof, although it will be understood that these examples are included merely for purposes of illustration and are not intended to limit the scope of the invention. Unless otherwise indicated, all percentages are by weight.

### EXAMPLES

### Example 1

### Preparation of Methyl Cocoate (Compound of Formula 2)

To a jar was added potassium hydroxide (1 g) and methanol (25 g). The solution was stirred for 1 hour. To a separate jar was added coconut oil (100 g). The solid was heated to a melt and the KOH/MeOH solution was added and the mixture was stirred overnight. The mixture was transferred to a separatory funnel and allowed to separate. The bottom/glycerol layer was removed. The top layer was filtered to afford a pale yellow oil (100 g). ¹H NMR (300 MHz, CDCl₃) δ 3.65 (s, 3H), 2.28 (t, 2H), 1.60 (t, 2H), 1.24 (s, 16H), 0.86 (t, 3H).

### Example 2

### Preparation of Dimethylaminoethyl Cocoate (Compound of Formula 4)

To a 1 L jacketed flask was added dimethylaminoethanol (98.05 g, 1.1 mol, 1.25 eq), Novozym 435 (4 g, 2 wt %) and methyl cocoate (200 g, 0.88 mol). The jacket was set at 50° C. and the mixture was stirred vigorously. The reaction mixture was sparged with nitrogen (ca. 0.5 L/min). The reaction was monitored by GCMS and NMR. The reaction was completed in 18 hours. The product was filtered to afford a pale yellow oil. ¹H NMR (300 MHz, CDCl₃) δ 4.15 (t, 2H), 2.54 (t, 2H), 2.31 (t, 2H), 2.26 (s, 6H), 1.60 (t, 2H), 1.24 (s, 16H), 0.86 (t, 3H).

### Example 3

### Preparation of Trimethylammoniumethyl Cocoate Methosulfate (Compound of Formula 1)

To a 100 mL flask was added dimethylaminoethyl cocoate (10 g, 0.035 mol) and acetone (20 mL). The flask was cooled to 0-10° C. in an ice water bath. To the flask was added dimethyl sulfate (4.41 g, 0.035 mol, 1 eq), slowly via addition funnel. The addition rate was such that the reaction temperature was kept below 25° C. When the addition was completed, the reaction was allowed to come to room temperature. When the reaction was completed as determined by NMR, the acetone was removed in vacuo to afford the product as a waxy white solid (14.8 g). ¹H NMR (300 MHz, DMSO d-6) δ 4.43 (m, 2H), 3.65 (m, 2H), 3.11 (s, 9H), 2.34 (t, 2H), 1.53 (t, 2H), 1.24 (s, 16H), 0.86 (t, 3H).

### Example 4

### Preparation of Trimethylammoniumethyl Cocoate Iodide (Compound of Formula 1)

To a 100 mL flask fitted with a condenser, a nitrogen bubbler and an addition funnel was added dimethylaminoethyl cocoate (10 g, 0.035 mol) and isopropanol (10 g). The flask was cooled in an ice water bath. To the solution was added methyl iodide (4.96 g, 0.035 mol, 1 eq) in isopropanol (10 g), slowly via addition funnel. The temperature was kept below 20° C. Additional isopropanol (20 g) was added to aid in stirring. When the addition was completed, the mixture was allowed to come to room temperature. A slight exotherm (ca. 10° C.) was observed upon warming. The milky reaction mixture was warmed to 40° C.; NMR revealed that the reaction was completed and that all methyl iodide was consumed. The reaction mixture was concentrated in vacuo to afford a white solid (14 g). ¹H NMR (300 MHz, DMSO d-6) δ 4.43 (m, 2H), 3.65 (m, 2H), 3.11 (s, 9H), 2.34 (t, 2H), 1.53 (t, 2H), 1.24 (s, 16H), 0.86 (t, 3H).

### Example 5

### Preparation of Dimethylethylammoniumethyl Cocoate Iodide (Compound of Formula 1)

To a 100 mL flask with condenser and addition funnel under nitrogen was added dimethylaminoethyl cocoate (10 g, 0.035 mol) and acetone (10 mL). The flask was cooled in an ice water bath. To the flask was added ethyl iodide (5.46 g, 0.035 mol) slowly via addition funnel. The temperature was below 10° C. When the addition was completed, the ice water bath was removed and the flask was allowed to come to room temperature. When the reaction was completed as determined by NMR, the acetone was removed in vacuo to afford the product as a waxy solid (15 g). ¹H NMR (300 MHz, DMSO d-6) δ 4.42 (m, 2H), 3.60 (m, 2H), 3.48 (q, 2H), 3.35 (t, 3H), 3.04 (s, 6H), 2.33 (t, 2H), 1.52 (t, 2H), 1.23 (s, 16H), 0.85 (t, 3H).

### Example 6

### Preparation of Benzyldimethylammoniumethyl Cocoate Chloride (Compound of Formula 1)

To a 100 mL flask was added dimethylaminoethyl cocoate (20 g, 0.0705 mol). To the flask was added benzyl chloride (8.93 g, 0.0705 mol, 1 eq) slowly. The reaction mixture was heated to 45° C. A ten degree exotherm (to 55° C.) was observed. The mixture became viscous and acetone was added to aid in stirring. When the reaction was completed as determined by NMR, the acetone was removed in vacuo to afford a viscous oil (24 g). ¹H NMR (300 MHz, DMSO d-6) δ 7.54 (m, 5H), 4.66 (s, 2H), 4.53 (m, 2H), 3.68 (m, 2H), 3.04 (s, 6H), 2.35 (t, 2H), 1.53 (t, 2H), 1.23 (s, 16H), 0.85 (t, 3H).

### Example 7

### Preparation of Bis(Cocoylethyl) Methylamine (Compound of Formula 4)

To a 1 L jacketed flask was added methyldiethanolamine (26.3 g, 0.22 mol), Novozym 435 (2 g, 2 wt %) and methyl cocoate (100 g, 0.44 mol). The jacket was set at 55° C. and the mixture was stirred vigorously. The reaction mixture was sparged with nitrogen (ca. 0.5 L/min). The reaction was monitored by NMR. The reaction was completed in 18 hours. The product was filtered to afford a pale yellow oil (99 g). ¹H NMR (300 MHz, CDCl₃) δ 4.17 (t, 4H), 2.71 (t, 4H), 2.36 (s, 3H), 2.31 (t, 4H), 1.62 (t, 4H), 1.26 (s, 32H), 0.88 (t, 6H).

### Example 8

### Preparation of Bis(Cocoylethyl)Dimethylammonium Methosulfate (Compound of Formula 1)

To a 100 mL flask was added bis(cocoylethyl)methylamine (20 g, 0.039 mol) with stirring at room temperature. To the flask was added dimethyl sulfate (4.96 g, 0.039 mol) slowly via addition funnel. The reaction was exothermic. Isopropanol (20 g) was added as the reaction mixture became viscous. The mixture was stirred for one hour at 60° C. The reaction was completed as determined by NMR. The isopropanol was removed in vacuo to afford the product as a white solid (24.2 g). ¹H NMR (300 MHz, DMSO d-6) δ 4.44 (m, 4H), 3.72 (m, 4H), 3.13 (s, 6H), 2.33 (t, 4H), 1.53 (t, 4H), 1.24 (s, 32H), 0.85 (t, 3H).

### Example 9

### Preparation of Bis(Cocoylethyl) 2-Hydroxyethylamine (Compound of Formula 4)

To a 1 L jacketed flask was added triethanolamine (32.82 g, 0.22 mol), Novozym 435 (2 g, 2 wt %) and methyl cocoate (100 g, 0.44 mol). The jacket was set at 55° C. and the mixture was stirred vigorously. The reaction mixture was sparged with nitrogen (ca. 0.5 L/min). The reaction was monitored by NMR. The reaction was completed in 18 hours. The product was filtered to afford a pale yellow oil (105 g). ¹H NMR (300 MHz, CDCl₃) δ 4.14 (m, 4H), 3.58 (m, 2H), 2.83 (m, 4H), 2.67 (m, 2H), 2.30 (m, 4H), 1.61 (t, 4H), 1.25 (s, 32H), 0.88 (t, 6H).

### Example 10

### Preparation of Methyl Bis(Cocoylethyl) (2-Hydroxyethyl)Ammonium Methosulfate (Compound of Formula 1)

To a 100 mL flask was added bis(cocoylethyl) 2-hydroxyethylamine (20 g, 0.037 mol) with stirring at room temperature. To the flask was added dimethyl sulfate (4.69 g, 0.037 mol) slowly via addition funnel. The reaction was exothermic. Isopropanol (20 g) was added as the reaction mixture became viscous. The mixture was stirred for one hour at 60° C. The reaction was completed as determined by NMR. The isopropanol was removed in vacuo to afford the product as a viscous, clear semi-solid (22.2 g). ¹H NMR (300 MHz, DMSO d-6) δ 4.44 (m, 4H), 3.84 (m, 2H), 3.78 (m, 4H), 3.53 (m, 2H), 3.15 (m, 3H), 2.32 (t, 4H), 1.52 (m, 4H), 1.24 (s, 32H), 0.85 (t, 6H).

### Example 11

The surfactant properties of the ester quats of general formula 1 can be determined by a number of tests including an ASTM foam height test and a test for critical micelle concentration.

The Standard Test Method for Foaming Properties of Surface-Active Agents (ASTM 1173-07) was used to determine the foaming properties of the ester quats 1 described herein. This method generates foam under low-agitation conditions and is generally used for moderate- and high-foam surfactants. This test gathers data on initial foam height and foam decay. Foam decay provides information on foam stability.

The apparatus for carrying out this test included a jacketed column and a pipet. The jacketed column served as a receiver, while the pipet delivers the surface-active solution.

Solutions of each surface-active agent from Examples 3-6, 8, and 10 were prepared. The ester quat solution to be tested was added to the receiver (50 mL) and to the pipet (200 mL). The pipet was positioned above the receiver and opened. As the solution from the pipet fell and made contact with the solution in the receiver, foam was generated. When the pipet was empty, the time was noted and an initial foam height was recorded. The foam height was recorded each minute for five minutes. Exact size specifications for the glassware can be found in ASTM 1173-07.

Data from the foam height test can be found in Table 1. These compounds were prepared at 1 g/L and 10 g/L aqueous solutions. As the data in Table 1 indicate, solutions of the ester quats generated large amounts of foam. Examples in which foam height does not decrease over time indicate good foam stability.

TABLE 1

| Surfactant | | | | | | | | | | | | |
|---|---|---|---|---|---|---|---|---|---|---|---|---|
| from | Foam height (cm) at time t (min) | | | | | | | | | | | |
| Example | 1 g/L (0.1 weight %) | | | | | | 10 g/L (1.0 weight %) | | | | | |
| No. | t = 0 | 1 | 2 | 3 | 4 | 5 | t = 0 | 1 | 2 | 3 | 4 | 5 |
| 3 | 18 | 18 | 18 | 18 | 18 | 18 | 16.5 | 16.5 | 16.5 | 16.5 | 16.5 | 16.5 |
| 4 | 17.5 | 17.5 | 17.5 | 17 | 17 | 17 | 17 | 17 | 17 | 17 | 16.5 | 16.5 |
| 5 | 17.5 | 17.5 | 17.5 | 17 | 17 | 17 | 16.5 | 16.5 | 16.5 | 16.5 | 16.5 | 16 |
| 6 | 17 | 17 | 16.5 | 15.5 | 15 | 14.5 | 15.5 | 15 | 14 | 13 | 11 | 4 |
| 8 | 11 | 11 | 11 | 11 | 11 | 11 | 14 | 14 | 14 | 14 | 14 | 14 |
| 10 | 8 | 8 | 8 | 8 | 8 | 8 | 11 | 11 | 11 | 11 | 11 | 11 |

The critical micelle concentration (CMC) was also determined for each compound. The CMC is the concentration of surfactants above which micelles spontaneously form. CMC is an important characteristic of a surfactant. At surfactant concentrations below the CMC, surface tension varies widely with surfactant concentration. At concentrations above the CMC, surface tension remains fairly constant. A lower CMC indicates less surfactant is needed to saturate interfaces and form micelles. Typical CMC values are less than 1 weight %.

The fluorimetric determination of CMC described by Chattopadhyay and London (Analytical Biochemistry, 139, 408-412, 1984) was used to obtain the critical micelle concentrations reported in Table 2. This method employs the fluorescent dye 1,6-diphenyl-1,3,5-hexatriene (DPH) in a solution of the surface-active agent. The analysis is based on differences in fluorescence upon incorporation of the dye into the interior of the micelles. As the solution exceeds CMC, a large increase in fluorescence intensity is observed. This method has been found to be sensitive and reliable, and has been demonstrated on zwitterionic, anionic, cationic, and uncharged surface-active agents.

TABLE 2

| Surfactant from | CMC |
|---|---|
| Example No. | (weight %) |
| 3 | 0.000261 |
| 4 | 0.005082 |
| 5 | 0.000609 |
| 6 | 0.000242 |
| 8 | 0.0000386 |
| 10 | 0.000118 |

The data in Table 2 indicate that very low concentrations of the ester quats in aqueous solutions are needed to reach CMC. As with foam height, all of these compounds appear similar. These values fall in the range of being useful as surface-active agents.

The invention has been described in detail with particular reference to preferred embodiments thereof, but it will be understood that variations and modifications can be effected within the spirit and scope of the invention.

We claim:

1. A process for preparing quaternary ammonium ester compounds, comprising:
(a) contacting a fatty acid ester with an amino-alcohol in the presence of an enzyme under conditions effective to form an amino-ester compound; and
(b) contacting the amino-ester compound with a quaternizing agent selected from alkyl halides, alkyl sulfates, alkyl phosphates, and alkyl carbonates under conditions effective to produce a quaternary ammonium ester compound.

2. The process according to claim 1, wherein the quaternary ammonium ester compound has the general formula 1: 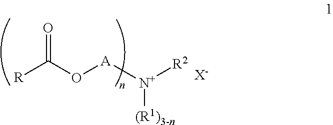
wherein
R is selected from substituted and unsubstituted, branched- and straight-chain, saturated, unsaturated, and polyunsaturated C₁-C₂₂ hydrocarbyl, substituted and unsubstituted C₃-C₈ cycloalkyl, substituted and unsubstituted C₈-C₂₀ carbocyclic aryl, and substituted and unsubstituted C₄-C₂₀ heterocyclic wherein the heteroatoms are selected from sulfur, nitrogen, and oxygen, and mixtures thereof;
R¹ and R², which may be the same or different, are independently selected from substituted or unsubstituted straight- or branched-chain C₁-C₂₂ alkyl, alkenyl, dienyl, trienyl, and C₃-C₈ cycloalkyl groups wherein the branching and/or substitution of R¹ and R² may connect to form a ring;
A is selected from substituted and unsubstituted, branched- and straight-chain, saturated, unsaturated, and polyunsaturated C₁-C₁₀ divalent hydrocarbyl, substituted and unsubstituted C₃-C₈ cycloalkylene, substituted and unsubstituted C₆-C₁₀ carbocyclic arylene, and substituted and unsubstituted C₄-C₁₀ divalent heterocyclic wherein the heteroatoms are selected from sulfur, nitrogen, and oxygen;
X⁻ is an anion of the quaternizing agent; and
n is an integer from 1 to 3.

3. The process according to claim 2, wherein the fatty acid ester has the general formula 2: 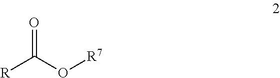
wherein R is as defined in formula 1, and R⁷ is a C₁-C₆ straight or branched chain alkyl.

4. The process according to claim 3, wherein the amino-alcohol has the general formula 3: 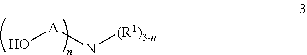
wherein R¹, A, and n are as defined in formula 1.

5. The process according to claim 4, wherein the amino-ester compound has the general formula 4: 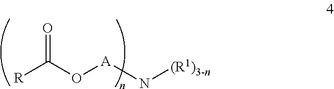
wherein R, R¹, A, and n are as defined in formula 1.

6. The process according to claim 2, wherein R is C₅ to C₁₇ hydrocarbyl; R¹ is methyl or hydroxyethyl; R² is methyl, ethyl, or benzyl; A is 1,2-ethylene or 1,3-propylene; and k is a halide, methylsulfate, ethylsulfate, carbonate, methyl carbonate, or phosphate.

7. The process according to claim 1, wherein step (a) is carried out at a temperature of 50 to 70° C. and a pressure of 1 kPa to 101 kPa.

8. The process according to claim 7, wherein the fatty acid ester is present in amount of 0.5 to 1.5 equivalents based on the number of hydroxyl groups on the amino-alcohol.

9. The process according to claim 8, wherein the enzyme is a lipase.

10. The process according to claim 1, wherein step (b) is carried out at a temperature of 0 to 100° C. and a pressure of 101 kPa to 1013 kPa.

11. The process according to claim 10, wherein the quaternizing agent is present in an amount of 0.9 to 1.5 equivalents based on the amino-ester compound.

12. The process according to claim 1, wherein the fatty acid ester is produced by reacting a triglyceride with a C₁-C₄ alcohol in the presence of a base.

13. The process according to claim 1, wherein step (a) is carried out in the absence of added solvent.

14. The process according to claim 1, wherein step (b) is carried out in the absence of added solvent.

15. The process according to claim 1, wherein step (b) is carried out in a solvent selected from water, alcohols, and ketones.

16. The process according to claim 15, wherein the solvent is isopropanol or acetone.

17. The process according to claim 1, wherein the quaternizing agent is methyl halide, ethyl halide, benzyl halide, or dimethyl sulfate.
